(19) United States
(12) Patent Application Publication
(10) Pub. No.: US 2021/0401327 A1
(43) Pub. Date: Dec. 30, 2021

(54) IMAGING APPARATUS, INFORMATION PROCESSING APPARATUS, IMAGE PROCESSING SYSTEM, AND CONTROL METHOD

(71) Applicant: CANON KABUSHIKI KAISHA, Tokyo (JP)

(72) Inventors: Yosato Hitaka, Tokyo (JP); Yoshikazu Kawai, Tokyo (JP)

(21) Appl. No.: 17/470,645

(22) Filed: Sep. 9, 2021

Related U.S. Application Data
(63) Continuation of application No. PCT/JP2020/008448, filed on Feb. 28, 2020.

(30) Foreign Application Priority Data
Mar. 12, 2019 (JP) 2019-045041
Feb. 14, 2020 (JP) 2020-023400

(51) Int. Cl. A61B 5/11; A61B 5/00

(52) U.S. Cl. CPC A61B 5/1116; A61B 5/0013; A61B 5/447; A61B 5/742

(57) ABSTRACT

The present invention is directed to enabling the capturing of an image to facilitate the comparison between affected parts. An imaging apparatus includes an image capturing unit, and a control unit configured to perform control to, in a case where posture information regarding an object obtained when an image of an affected part of the object has been captured in the past is acquired and the image capturing unit captures an image of the affected part of the object, notify a user of the posture information regarding the object and inclination information regarding the imaging apparatus.

### CROSS-REFERENCE TO RELATED APPLICATIONS

This application is a Continuation of International Patent Application No. PCT/JP2020/008448, filed Feb. 28, 2020, which claims the benefit of Japanese Patent Applications No. 2019-045041, filed Mar. 12, 2019, and No. 2020-023400, filed Feb. 14, 2020, all of which are hereby incorporated by reference herein in their entirety.

### BACKGROUND OF THE INVENTION

### Field of the Invention

The present invention relates to an imaging apparatus, an information processing apparatus, an image processing system, and a control method.

### Background Art

In a state where a person or an animal lies down, a part of the body in contact with a contact surface may be compressed by body weight, thereby developing a pressure ulcer, i.e., a bedsore. To a patient who has developed a pressure ulcer, it is necessary to provide pressure ulcer care such as body pressure dispersion care and skin care. Then, it is necessary to periodically evaluate and manage the pressure ulcer.

On page 23 of Shorinsha, Guidebook for Pressure Ulcers (second edition), in compliance with Guidelines for the Prevention and Management of Pressure Ulcers (fourth edition) (edited by the Japanese Society of Pressure Ulcers, ISBN13 978-4796523608), DESIGN-R (registered trademark), which is a pressure ulcer status determination scale developed by the Scientific Education Committee of the Japanese Society of Pressure Ulcers, is discussed as a tool for pressure ulcer evaluation. DESIGN-R is a tool for evaluating a healing process of a wound such as a pressure ulcer. The name of the scale is an acronym of Depth, Exudate, Size, Inflammation/Infection, Granulation, and Necrotic tissue, which are observation items.

DESIGN-R includes two types, namely DESIGN-R for classification of severity for daily simple evaluation, and DESIGN-R for progress evaluation indicating steps in the healing process in detail. DESIGN-R for classification of severity classifies six evaluation items into two levels, namely mild and severe levels. The mild level is represented using lowercase alphabetic characters, and the severe level is represented using uppercase alphabetic characters.

A pressure ulcer is evaluated using the classification of severity in an initial treatment, whereby it is possible to grasp a general state of the pressure ulcer. It identifies which item is problematic, and thus, a treatment strategy can be easily determined.

Meanwhile, as DESIGN-R for progress evaluation, DESIGN-R capable of comparing severity between patients in addition to providing the progress evaluation is also defined. R represents rating (evaluation or grading). Each item is weighted differently, and a total point (0 to 66 points) of the six items except for Depth indicate the severity of the pressure ulcer. After the treatment is started, progress of the treatment can be evaluated in detail and objectively. Thus, it is possible not only to evaluate the progress for an individual, but also to compare the severity between patients.

In Size evaluation of DESIGN-R, Size is classified into seven levels, where Size is a numerical value obtained by measuring, in centimeters, the major axis and the minor axis (the maximum diameter orthogonal to the major axis) of the extent of skin injury and by multiplying the major axis and the minor axis. The seven levels are: s0 indicating no skin injury, s3 indicating Size is less than 4, s6 indicating Size is 4 or more and less than 16, s8 indicating Size is 16 or more and less than 36, s9 indicating Size is 36 or more and less than 64, s12 indicating Size is 64 or more and less than 100, and s15 indicating Size is 100 or more.

In scoring of DESIGN-R, as discussed in Guidebook for Pressure Ulcers, it is recommended that scoring be performed once a week or two weeks to evaluate progress in healing of the pressure ulcer and select appropriate care. Thus, it is necessary to periodically evaluate and manage a medical condition of the pressure ulcer. Accuracy is required in the evaluation to check a change in the medical condition of the pressure ulcer.

### CITATION LIST

### Non-Patent Literature

NPL 1: Japanese Society of Pressure Ulcers. (2015), Guidebook for Pressure Ulcers (second edition): in compliance with Guidelines for the Prevention and Management of Pressure Ulcers (fourth edition). Shorinsha.

### SUMMARY OF THE INVENTION

However, in a case where a pressure ulcer is captured, the shape, the area, and the shape of a pocket of the pressure ulcer change depending on posture of the patient. Thus, every time the pressure ulcer is captured, visibility of the pressure ulcer may change. Thus, it has been difficult to accurately compare the progress of the pressure ulcer by comparing images obtained by capturing the pressure ulcer. This is not limited to a pressure ulcer, and the same applies to a case where a burn or a laceration is captured.

The present invention is directed to enabling the capturing of an image to facilitate the comparison between affected parts.

According to an aspect of the present invention, an imaging apparatus includes an image capturing unit, and a control unit configured to perform control to, in a case where posture information regarding an object obtained when an image of an affected part of the object has been captured in the past is acquired and the image capturing unit captures an image of the affected part of the object, notify a user of the posture information regarding the object and inclination information regarding the imaging apparatus.

Further features of the present invention will become apparent from the following description of exemplary embodiments with reference to the attached drawings.

### BRIEF DESCRIPTION OF THE DRAWINGS

FIG. 1 is a diagram illustrating a functional configuration of an image processing system.

FIG. 2 is a diagram illustrating an object.

FIG. 3 is a diagram illustrating a hardware configuration of an imaging apparatus.

FIG. 4 is a diagram illustrating a hardware configuration of an information processing apparatus.

FIG. 5, which includes FIGS. 5A and 5B, is a flowchart illustrating processing of the image processing system.

FIG. 6 is a diagram illustrating a calculation method for calculating an area of an affected region.

FIG. 7A is a diagram illustrating a method for superimposing information on image data of an affected part.

FIG. 7B is a diagram illustrating a method for superimposing information on image data of an affected part.

FIG. 8A is a diagram illustrating a method for superimposing information on image data of an affected part.

FIG. 8B is a diagram illustrating a method for superimposing information on image data of an affected part.

FIG. 8C is a diagram illustrating a method for superimposing information on image data of an affected part.

FIG. 9A is a diagram illustrating a data configuration of object information.

FIG. 9B is a diagram illustrating a data configuration of object information.

FIG. 10A is a diagram illustrating image data including posture information.

FIG. 10B is a diagram illustrating image data including posture information.

### DESCRIPTION OF THE EMBODIMENTS

Exemplary embodiments of the present invention will be described below with reference to the drawings.

### First Exemplary Embodiment

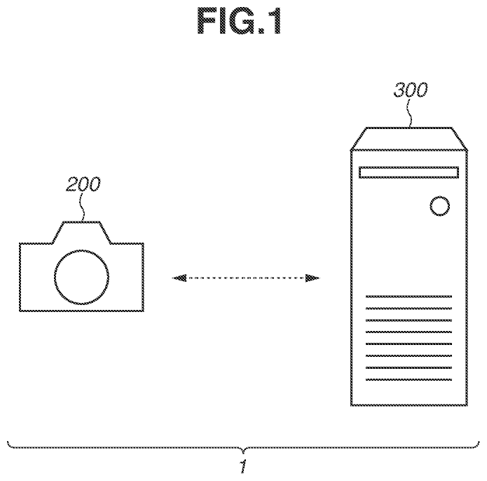

FIG. 1 is a diagram illustrating an example of a functional configuration of an image processing system 1.

The image processing system 1 includes an imaging apparatus 200 that is a handheld portable device, and an information processing apparatus 300.

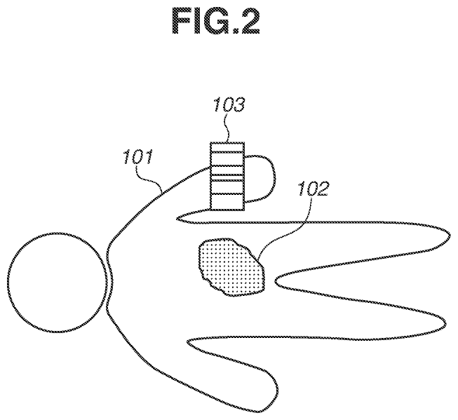

FIG. 2 is a diagram illustrating an example of an object 101 that is a patient whose affected part is evaluated by the image processing system 1. In the present exemplary embodiment, as an example of a clinical condition of an affected part 102 developed in the buttocks of the object 101, a pressure ulcer developed in the buttocks is described.

A barcode tag 103 is attached to the object 101. The barcode tag 103 includes patient identification (ID) as identification information for identifying the object 101. Thus, the image processing system 1 can manage the identification information regarding the object 101 and image data obtained by capturing an image of the affected part 102 in association with each other. The identification information is not limited to the barcode tag 103, and may be a two-dimensional code such as a QR code (registered trademark) or a numerical value, or may be data or an ID number attached to an ID card such as a patient registration card.

In the image processing system 1, the imaging apparatus 200 captures images of the affected part 102 of the object 101 and the barcode tag 103 as the identification information and transmits the images to the information processing apparatus 300. The information processing apparatus 300 transmits posture information regarding the object 101 obtained when an image of the affected part 102 of the same object 101 has been captured in the past, as posture information associated with the received identification information to the imaging apparatus 200. The imaging apparatus 200 performs display based on the received posture information, whereby a user can grasp the posture of the object 101 taken when the image of the affected part 102 of the same object 101 has been captured in the past. The posture information may only need to include at least information that allows identification of the posture of the object 101 to be any one of a prone posture, a recumbent posture (a right lateral recumbent posture or a left lateral recumbent posture), and a sitting posture. While the present exemplary embodiment is described using an example in which the affected part 102 is a pressure ulcer, the affected part 102 is not limited to a pressure ulcer and may be a burn or a laceration.

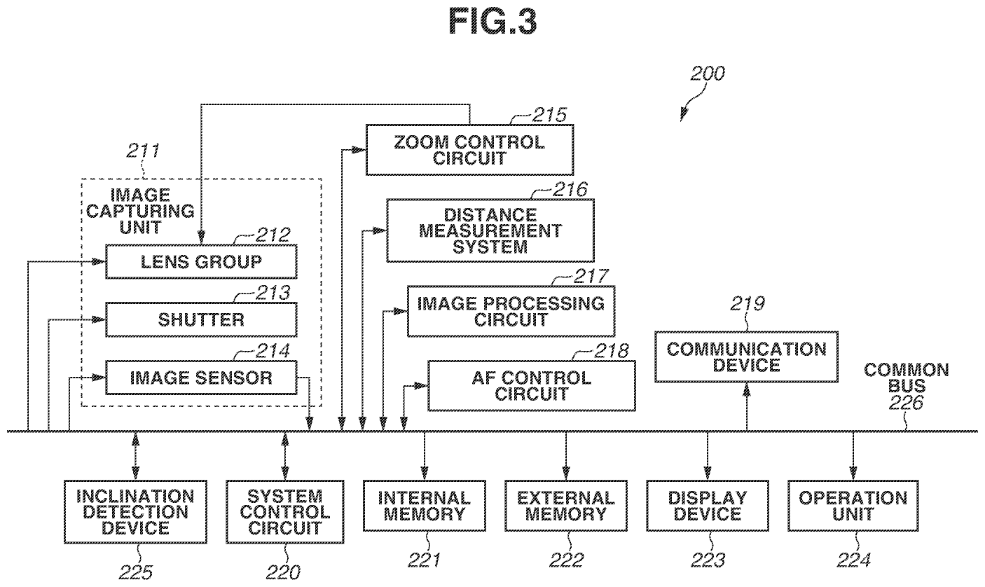

FIG. 3 is a diagram illustrating an example of a hardware configuration of the imaging apparatus 200.

As the imaging apparatus 200, a general single-lens reflex camera, a compact digital camera, or a smartphone or a tablet terminal including a camera having an autofocus function can be used.

An image capturing unit 211 includes a lens group 212, a shutter 213, and an image sensor 214. By changing positions of a plurality of lenses included in the lens group 212, a focus position and a zoom magnification can be changed. The lens group 212 also includes a diaphragm for adjusting an amount of exposure.

The image sensor 214 is composed of a charge accumulation type solid state image sensor such as a charge-coupled device (CCD) sensor or a complementary metal-oxide-semiconductor (CMOS) sensor, which converts an optical image into electric data. Reflected light from the object 101 having passed through the lens group 212 and the shutter 213 forms an image on the image sensor 214. The image sensor 214 generates an electric signal corresponding to an object image and outputs image data based on the generated electric signal.

The shutter 213 performs operation of opening and closing a blade member, thereby exposing the image sensor 214 and blocking light from reaching the image sensor 214. Thus, the shutter 213 controls an exposure time of the image sensor 214. The shutter 213 may be an electronic shutter that controls the exposure time by driving the image sensor 214. In a case where the electronic shutter is implemented using a CMOS sensor, a reset scan for setting an amount of accumulated charge of each pixel or an amount of accumulated charge of pixels in each region (e.g., each line) including a plurality of pixels to zero is performed. Then, for each pixel or each region subjected to the reset scan, after a predetermined time elapses, scanning for reading a signal corresponding to the amount of accumulated charge is performed.

A zoom control circuit 215 controls a motor for driving a zoom lens included in the lens group 212, thereby controlling an optical magnification of the lens group 212.

A distance measurement system 216 calculates distance information regarding a distance to the object 101. The distance measurement system 216 may generate the distance information based on output of an autofocus (AF) control circuit 218. In a case where there is a plurality of areas to be AF targets in a screen, the distance measurement system 216 may cause the AF control circuit 218 to repeatedly perform an AF process on each area, thereby generating the distance information for each area. The distance measurement system 216 may use a time-of-flight (ToF) sensor. The TOF sensor is a sensor that measures a distance to a physical body based on a time difference (or a phase difference) between a transmission timing of an irradiation wave and a reception timing of a reflected wave of the irradiation wave reflected by the physical body. Further, the distance measurement system 216 may use a position sensitive device (PSD) method using a PSD as a light-receiving element.

An image processing circuit 217 performs predetermined image processing on the image data output from the image sensor 214. The image processing circuit 217 performs various types of image processing such as white balance adjustment, gamma correction, color interpolation, demosaicing, and filtering on image data output from the image capturing unit 211 or image data stored in an internal memory 221. The image processing circuit 217 also performs a compression process based on a standard such as the Joint Photographic Experts Group (JPEG) standard on the image data subjected to the image processing.

Based on the distance information obtained by the distance measurement system 216, the AF control circuit 218 determines the position of a focus lens included in the lens group 212 and controls a motor for driving the focus lens. The AF control circuit 218 may perform TV-AF, or contrast AF, for extracting and integrating a high-frequency component of the image data and determining a position of the focus lens at which the integral value is the greatest. The focus control method is not limited to the contrast AF and may be phase difference AF or another AF method. Further, the AF control circuit 218 may detect an amount of focus adjustment or a position of the focus lens and, based on the position of the focus lens, acquire distance information regarding the distance to the object 101.

A communication device 219 is a communication interface for communicating with an external device such as the information processing apparatus 300 using a wireless network. Specific examples of the network include a network based on the Wi-Fi (registered trademark) standard. Communication using Wi-Fi may be implemented via a router. Alternatively, the communication device 219 may be implemented by a wired communication interface based on the Universal Serial Bus (USB) standard or the local area network (LAN) standard.

A system control circuit 220 includes a central processing unit (CPU). The system control circuit 220 executes a program stored in the internal memory 221, thereby controlling the entire imaging apparatus 200. The system control circuit 220 also controls the image capturing unit 211, the zoom control circuit 215, the distance measurement system 216, the image processing circuit 217, and the AF control circuit 218. The system control circuit 220 is not limited to a configuration including a CPU and may include a field-programmable gate array (FPGA) or an application-specific integrated circuit (ASIC).

As the internal memory 221, for example, a rewritable memory such as a flash memory or a synchronous dynamic random-access memory (SDRAM) can be used. The internal memory 221 temporarily stores various pieces of setting information required for the operation of the imaging apparatus 200, such as information regarding a focus position when an image is captured, the image data captured by the image capturing unit 211, and the image data subjected to the image processing by the image processing circuit 217. The internal memory 221 may temporarily store the image data and analysis data on information regarding the size of the object 101 that are received by the communication device 219 communicating with the information processing apparatus 300.

An external memory 222 is a non-volatile recording medium attachable to the imaging apparatus 200 or built into the imaging apparatus 200. As the external memory 222, for example, a Secure Digital (SD) card or a CompactFlash (CF) card can be used. The external memory 222 records the image data subjected to the image processing by the image processing circuit 217 and the image data and the analysis data received by the communication device 219 communicating with the information processing apparatus 300. When reproduction is performed, the image data recorded in the external memory 222 is read and can be output to outside the imaging apparatus 200.

As a display device 223, for example, a thin-film transistor (TFT) liquid crystal display, an organic electroluminescent (EL) display, or an electronic viewfinder (EVF) can be used. The display device 223 displays the image data temporarily stored in the internal memory 221 or the image data recorded in the external memory 222, or displays a setting screen for the imaging apparatus 200.

An operation unit 224 includes a button, a switch, a key, and a mode dial that are provided in the imaging apparatus 200 or a touch panel that also serves as the display device 223. The system control circuit 220 is notified of a command such as a mode setting or an image capturing instruction from the user via the operation unit 224.

An inclination detection device 225 detects an inclination of the imaging apparatus 200. In the present exemplary embodiment, the inclination of the imaging apparatus 200 refers to an angle based on the horizontal direction. As the inclination detection device 225, for example, a gyro sensor or an acceleration sensor can be used.

A common bus 226 is a signal line for transmitting and receiving a signal between the components of the imaging apparatus 200.

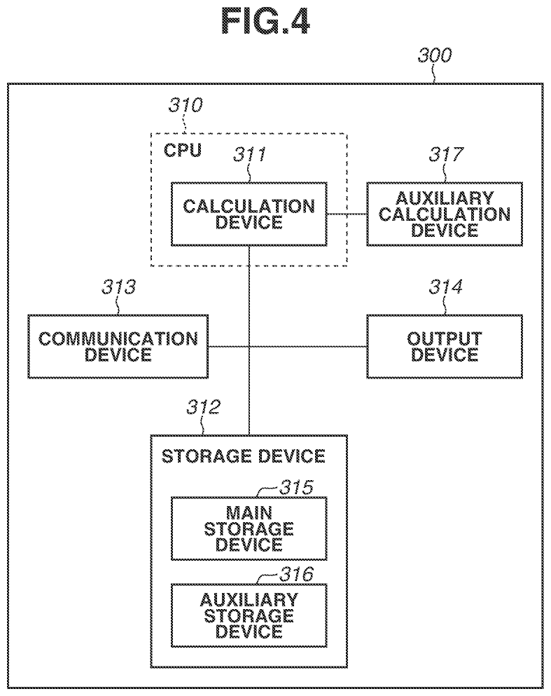

FIG. 4 is a diagram illustrating an example of a hardware configuration of the information processing apparatus 300.

The information processing apparatus 300 includes a CPU 310, a storage device 312, a communication device 313, an output device 314, and an auxiliary calculation device 317.

The CPU 310 includes a calculation device 311. The CPU 310 executes a program stored in the storage device 312, thereby controlling the entirety information processing apparatus 300 and also implementing the functional configuration of the information processing apparatus 300 illustrated in FIG. 4.

The storage device 312 includes a main storage device 315 (a read-only memory (ROM) or a random-access memory (RAM)) and an auxiliary storage device 316 (a magnetic disk device or a solid-state drive (SSD)).

The communication device 313 is a wireless communication module for communicating with an external device such as the imaging apparatus 200 using a wireless network.

The output device 314 outputs data processed by the calculation device 311 or data stored in the storage device 312 to a display, a printer, or an external network connected to the information processing apparatus 300.

The auxiliary calculation device 317 is an auxiliary calculation integrated circuit (IC) that operates under control of the CPU 310. As the auxiliary calculation device 317, a graphics processing unit (GPU) can be used. The GPU is originally an image processing processor, but can also be used as a processor that performs processing for signal learning because the GPU includes a plurality of product-sum calculators and excels at matrix calculations. Thus, the GPU is generally used in processing for performing deep learning. As the auxiliary calculation device 317, for example, Jetson TX2 Module manufactured by Nvidia Corporation can be used. Alternatively, as the auxiliary calculation device 317, an FPGA or an ASIC may be used. The auxiliary calculation device 317 performs an extraction process for extracting an affected region from image data.

The information processing apparatus 300 may include the single CPU 310 or a plurality of CPUs 310 and include the single storage device 312 or a plurality of storage devices 312. In other words, at least one or more CPUs and at least one or more storage devices are connected together, and if the at least one or more CPUs execute a program stored in the at least one or more storage devices, the information processing apparatus 300 executes functions described below. The information processing apparatus 300 may include not only the CPU but also an FPGA or an ASIC.

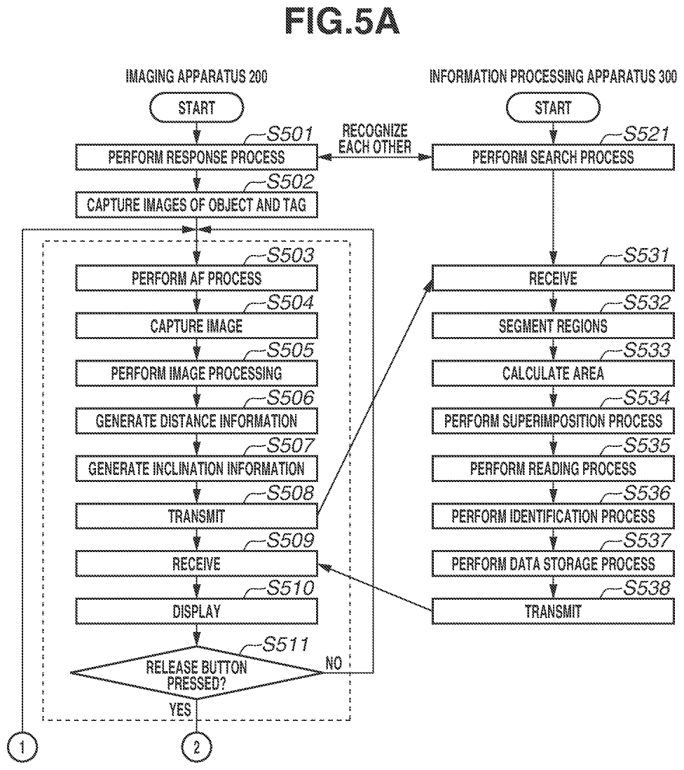

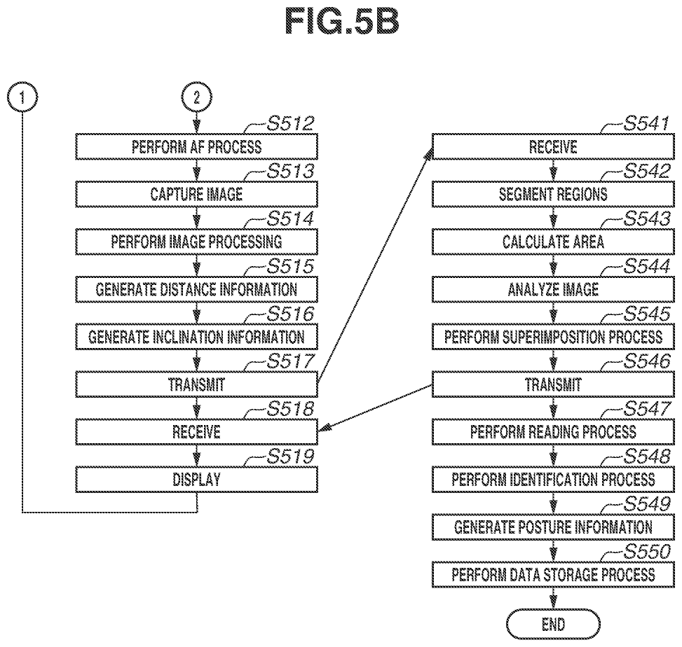

FIG. 5, which includes FIGS. 5A and 5B, is a flowchart illustrating an example of the processing of the image processing system 1.

In FIGS. 5A and 5B, steps S501 to S519 are the processing performed by the imaging apparatus 200, and steps S521 to S550 are the processing performed by the information processing apparatus 300. The flowchart in FIGS. 5A and 5B is started by the imaging apparatus 200 and the information processing apparatus 300 connecting to a network based on the Wi-Fi standard, which is a wireless LAN standard.

In step S521, the CPU 310 of the information processing apparatus 300 performs, via the communication device 313, a search process in search of the imaging apparatus 200 to connect to.

In step S501, the system control circuit 220 of the imaging apparatus 200 performs, via the communication device 219, a response process in response to the search process performed by the information processing apparatus 300. As a technique for searching for a device via a network, Universal Plug and Play (UPnP) is used. In PnP, an individual apparatus is identified by a universally unique identifier (UUID).

In step S502, using the display device 223, the system control circuit 220 prompts the user to capture an image of entire body posture of an object from which a posture of the object when an image of an affected part is captured can be understood, and an image of a barcode tag for identifying the object. In response to an image capturing instruction from the user, the image capturing unit 211 captures the images of the posture of the object and the barcode tag of the object.

In this step, before the image of the affected part of the object is captured, the object is asked to take, for example, a prone posture, a recumbent posture, or a sitting posture. Then, the user captures the image of the entire body posture of the object from which the posture of the object when the image of the affected part is captured can be understood. At this time, based on inclination information output from the inclination detection device 225, the system control circuit 220 generates inclination information regarding the imaging apparatus 200 when the posture is captured.

Next, processing for live view in steps S503 to S511 is described.

In step S503, the AF control circuit 218 performs an AF process for controlling the driving of the lens group 212 so that the object comes into focus.

In this step, since it is assumed that the user holds the imaging apparatus 200 so that the affected part is at the center of the screen, the AF control circuit 218 performs the AF process in an area at the center of the screen. Based on the amount of focus adjustment or the amount of movement of the focus lens, the AF control circuit 218 outputs the distance information regarding the distance to the object.

In step S504, using the display device 223, the system control circuit 220 prompts the user to capture an image of the affected part of the object. In response to an image capturing instruction from the user, the image capturing unit 211 captures an image of the object.

In step S505, the image processing circuit 217 acquires data of the captured image and performs a development process and a compression process on the image data, thereby generating image data based on the JPEG standard, for example. The image processing circuit 217 performs a resizing process on the image data subjected to the compression process, thereby reducing the size of the image data.

The imaging apparatus 200 will transmit the image data subjected to the resizing process using wireless communication in step S508 described below. The larger the size of the image data to be transmitted is, the longer time it takes to perform the wireless communication. Thus, in step S505, based on an acceptable communication time, the system control circuit 220 determines the size of the image data to be subjected to the resizing process and gives an instruction to the image processing circuit 217.

In step S532 described below, the information processing apparatus 300 will extract an affected region from the image data subjected to the resizing process. The size of the image data influences a time taken to extract the affected region and accuracy of the extraction. Thus, in step S505, based on the time taken to extract the affected region and the accuracy of the extraction, the system control circuit 220 determines the size of the image data to be subjected to the resizing process.

The resizing process in step S505 is processing performed during the live view. Thus, if a processing time is long, a frame rate of live view images becomes low. Thus, in step S505, it is desirable that the system control circuit 220 perform the resizing process in which the image data is resized to a smaller size than or the same size as that in a resizing process in step S514 described below, which is not the processing performed during the live view.

In the present exemplary embodiment, the image data is resized to a size of approximately 1.1 megabytes in the case of 720 pixels×540 pixels in 8-bit red, green, and blue (RGB) colors. However, the size of the image data to be subjected to the resizing process is not limited to the above.

In step S506, the system control circuit 220 generates distance information regarding the distance to the object. Specifically, based on the distance information output from the AF control circuit 218, the system control circuit 220 generates the distance information regarding the distance from the imaging apparatus 200 to the object. If the AF control circuit 218 performs the AF process on each of a plurality of areas in the screen in step S503, the system control circuit 220 may generate the distance information with respect to each of the plurality of areas. As for a method for generating the distance information, the distance information regarding the distance to the object calculated by the distance measurement system 216 may be used.

In step S507, based on the inclination information output from the inclination detection device 225, the system control circuit 220 generates inclination information regarding the imaging apparatus 200 in the live view.

In this step, since it is assumed that the user holds the imaging apparatus 200 so that an image capturing range thereof includes the affected part, the system control circuit 220 generates inclination information regarding the imaging apparatus 200 when the user holds the imaging apparatus 200 and points the imaging apparatus 200 at the affected part.

In step S508, the system control circuit 220 transmits various pieces of information to the information processing apparatus 300 via the communication device 219. Specifically, the system control circuit 220 transmits the image data of the affected part subjected to the resizing process in step S505, the distance information regarding the distance to the object that is generated in step S506, and the inclination information regarding the imaging apparatus 200 in the live view that is generated in step S507. The system control circuit 220 also transmits image data of the posture captured in step S502, the inclination information regarding the imaging apparatus 200 when the posture is captured, and image data of the barcode tag to the information processing apparatus 300. Patient ID included in the image data of the barcode tag is not information that changes. Thus, regarding the same patient, the system control circuit 220 transmits the image data of the barcode tag only the first time. Regarding the same patient, the system control circuit 220 also transmits the image data of the posture and the inclination information regarding the imaging apparatus 200 when the posture is captured only once the first time.

Next, the processing performed by the information processing apparatus 300 will be described.

In step S531, the CPU 310 of the information processing apparatus 300 receives, via the communication device 313, the image data of the affected part, the distance information regarding the distance to the object, and the inclination information regarding the imaging apparatus 200 in the live view that are transmitted from the imaging apparatus 200. Regarding the same patient, the CPU 310 receives the image data of the posture, the inclination information regarding the imaging apparatus 200 when the posture is captured, and the image data of the barcode tag only the first time.

In step S532, using the auxiliary calculation device 317, the CPU 310 extracts an affected region from the received image data of the affected part (segments the affected region and another region). As a technique for the region segmentation, semantic region segmentation using deep learning is performed. More specifically, a learning computer is trained in advance on a model of a neural network using a plurality of images of affected regions of actual pressure ulcers as supervised data, thereby generating a trained model. The auxiliary calculation device 317 acquires the trained model from the computer and estimates a pressure ulcer area from the image data based on the trained model. As an example of the model of the neural network, a model of a fully convolutional network (FCN), which is a segmentation model using deep learning, can be applied. An inference of deep learning is processed by the GPU that is included in the auxiliary calculation device 317 and that excels at parallel execution of product-sum calculations. However, the inference of deep learning may be executed by an FPGA or an ASIC. Alternatively, the region segmentation may be implemented using another model of deep learning. The segmentation technique is not limited to the deep learning, and for example, graph cuts, region growing, edge detection, or a divide-and-conquer method may be used. Further, a model of the neural network may be trained within the auxiliary calculation device 317 using the images of affected regions of pressure ulcers as the supervised data.

In step S533, the calculation device 311 of the CPU 310 calculates the area of the affected region as information regarding the size of the extracted affected region. The calculation device 311 converts the size of the extracted affected region in the image data based on information regarding the angle of view or the pixel size of the image data and the distance information generated by the system control circuit 220, thereby calculating the area of the affected region.

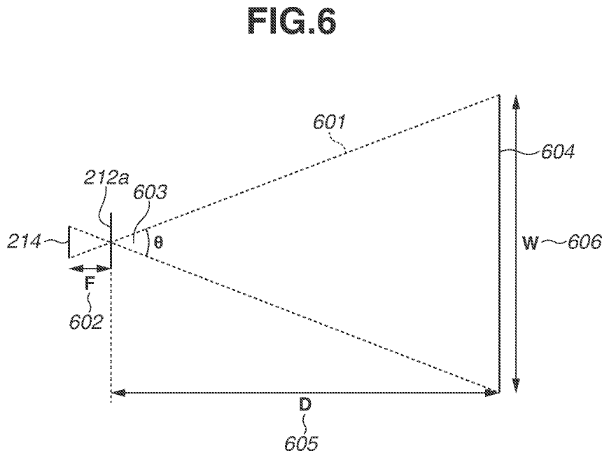

FIG. 6 is a diagram illustrating a calculation method for calculating the area of the affected region.

In a case where the imaging apparatus 200 is a general camera, the imaging apparatus 200 can be treated as a pinhole model as illustrated in FIG. 6. Incident light 601 passes through a lens principal point of a lens 212a and is received by an imaging surface of the image sensor 214. The distance from the imaging surface to the lens principal point is a focal length F602. In a case where the lens group 212 is approximated to the single lens 212a that is not thick, two principal points, namely a front principal point and a rear principal point, of the lens 212a can be regarded as coinciding with each other. The focus position of the lens 212a is adjusted so that an image is formed on a flat surface of the image sensor 214, whereby the imaging apparatus 200 can focus on an object 604. The focal length F602, which is the distance from the imaging surface to the lens principal point, is changed, thereby changing an angle of view θ603. This changes the zoom magnification. At this time, based on a relationship between the angle of view θ603 of the imaging apparatus 200 and an object distance D605, an object width W606 on a focal plane is geometrically determined. The object width W606 is calculated using a trigonometric function. More specifically, the object width W606 is determined based on the relationship between the angle of view θ603 that changes depending on the focal length F602, and the object distance D605. A value of the object width W606 is divided by the number of pixels on a corresponding line of the image sensor 214, thereby acquiring the length on the focal plane corresponding to one pixel on the image.

The calculation device 311 calculates the area of the affected region as the product of the number of pixels in the region obtained from a result of the region segmentation in step S532, and the area of one pixel obtained from the length on the focal plane corresponding to one pixel on the image. A formula for obtaining the object width W606 or the length on the focal plane corresponding to one pixel on the image may be recursively obtained by acquiring data while changing the object distance D605 and capturing an object of which the object width W606 is known.

In a case where the object distance D605 is a single distance, the calculation device 311 can correctly obtain the area of the affected region on the premise that the object 604 is a flat surface and the flat surface is perpendicular to the optical axis. If, however, the distance information is generated with respect to each of the plurality of areas in step S506, the calculation device 311 may detect the inclination of or a change in the object in the depth direction, and based on the detected inclination or change, calculate the area of the affected region.

In step S534, the image processing circuit 217 generates image data obtained by superimposing information indicating a result of extraction of the affected region and information regarding the size of the affected region on the image data from which the affected region is to be extracted.

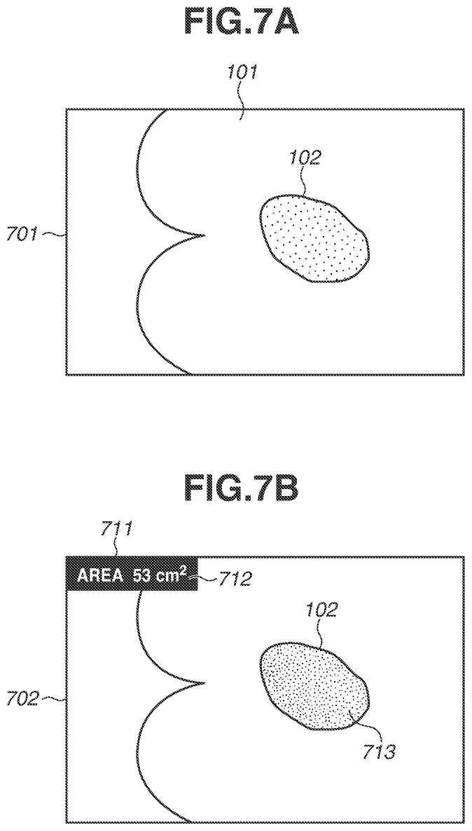

FIGS. 7A and 7B are diagrams illustrating a method for superimposing the information indicating the result of extraction of the affected region and the information regarding the size of the affected region on the image data.

An image 701 illustrated in FIG. 7A is an example of display of image data before the superimposition process is performed, and includes the object 101 and the affected part 102. An image 702 illustrated in FIG. 7B is an example of display of image data after the superimposition process is performed.

At the upper left corner of the image 702 illustrated in FIG. 7B, a label 711 is superimposed in which a character string 712 indicating the area of the affected region is displayed in white characters on a black background. In the image 702, the information regarding the size of the affected region is the character string 712 and is the area of the affected region calculated by the calculation device 311. The background color and the color of the character string of the label 711 are not limited to black and white as long as the colors are easy to see. Further, an amount of transmission may be set and alpha blending (a-blending) may be performed, so that the user can check the image in a portion where the label 711 is superimposed.

On the image 702, an indicator 713 indicating an estimated area of the affected region extracted in step S532 is superimposed. The indicator 713 indicating the estimated area and the image data from which the image 701 is generated are subjected to a-blending, whereby the user can check whether the estimated area from which the area of the affected region is calculated is appropriate. It is desirable that the color of the indicator 713 indicating the estimated area be different from the color of the object 101. It is also desirable that a range of transmittance of the a-blending be a range where the estimated area and the original affected part 102 can be distinguished from each other. If the indicator 713 indicating the estimated area of the affected region is displayed in a superimposed manner, the user can check whether the estimated area is appropriate even if the label 711 is not displayed. Thus, step S533 may be omitted.

In step S535, the CPU 310 reads the patient ID from the image data of the barcode tag. In step S536, the CPU 310 checks the read patient ID against the patient ID of the object registered in advance in the storage device 312, thereby acquiring information regarding the name of the object.

In step S537, the CPU 310 stores the image data of the affected part in association with the patient ID and the information regarding the name of the object in the storage device 312. Until the CPU 310 receives image data of a barcode tag that is subsequently captured, the CPU 310 processes the image data of the affected part received in step S531 as data regarding the same patient ID and the same information regarding the name of the object.

The CPU 310 also determines whether object information corresponding to the target patient ID is stored in the storage device 312. If the object information corresponding to the target patient ID is not stored, the CPU 310 generates object information corresponding to the patient ID and the information regarding the name of the object. On the other hand, if the object information corresponding to the target patient ID is already stored in the storage device 312, the processing proceeds to step S538.

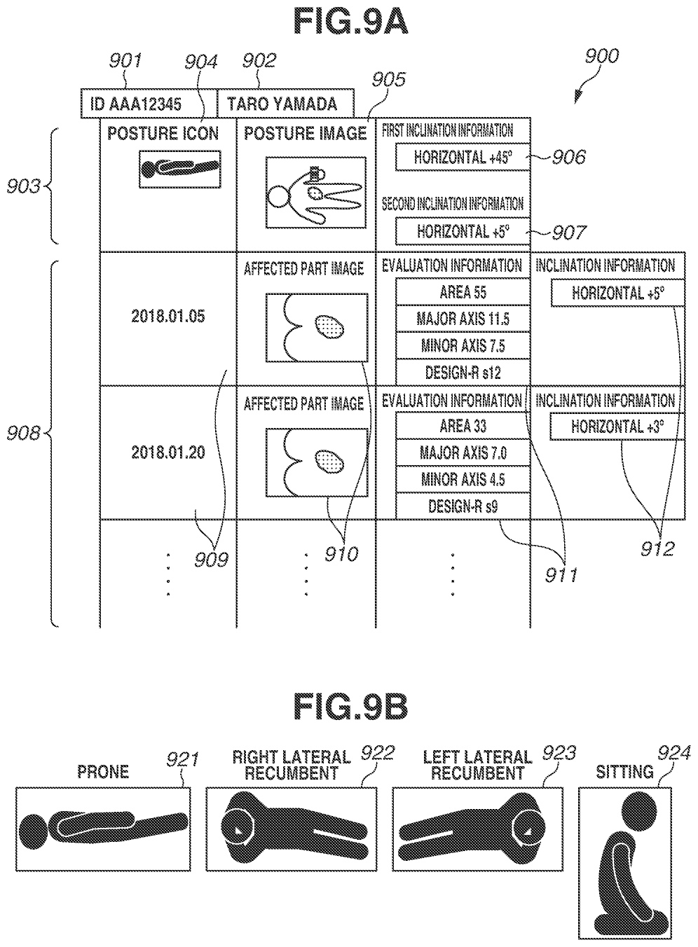

FIG. 9A is a diagram illustrating an example of a data configuration of object information 900. The object information 900 is managed with respect to each patient ID.

The object information 900 includes a patient ID field 901, a name-of-object field 902, posture information 903, and affected part information 908.

The patient ID field 901 stores the patient ID. The name-of-object field 902 stores the name of the object.

The posture information 903 includes a posture icon field 904, an image-data-of-posture field 905, a first inclination information field 906, and a second inclination information field 907. The posture icon field 904 stores a posture icon schematically illustrating the posture of the object when the image of the affected part is captured or identification information regarding the posture icon. The posture icon corresponds to an example of a display item.

FIG. 9B is a diagram illustrating examples of the posture icon.

A posture icon 921 is an icon representing a prone posture. A posture icon 922 is an icon representing a right lateral recumbent posture with the right side down. A posture icon 923 is an icon representing a left lateral recumbent posture with the left side down. A posture icon 924 is an icon representing a sitting posture.

The image-data-of-posture field 905 stores the image data of the posture obtained by capturing the posture of the object in step S502 or address information regarding an address where the image data of the posture is stored.

The first inclination information field 906 stores the inclination information regarding the imaging apparatus 200 when the posture is captured in step S502. The second inclination information field 907 stores inclination information regarding the imaging apparatus 200 in image capturing for recording in which the live view is ended and the image of the affected part is captured as a record. The second inclination information field 907 stores the inclination information regarding the imaging apparatus 200 when the image capturing for recording is performed for the first time or the last time with the target patient ID, or an average value of pieces of inclination information regarding the imaging apparatus 200 when the image capturing for recording is performed multiple times. The inclination information in the second inclination information field 907 is stored or updated based on inclination information regarding the imaging apparatus 200 in the image capturing for recording that is stored in an inclination information field 912. When performing the image capturing for recording, the user references the inclination information stored in the second inclination information field 907 and thereby can use the inclination information to cause the imaging apparatus 200 to face the surface of the affected part.

The posture information 903 may store information that allows identification of the posture of the object, such as character information representing the posture of the object in characters “prone”, “sitting”, “right lateral recumbent”, or “left lateral recumbent”.

The affected part information 908 includes an image-capturing-date-and-time field 909, an image-data-of-affected-part field 910, an evaluation information field 911, and the inclination information field 912. The image-capturing-date-and-time field 909 stores a date and time of the image capturing for recording performed in step S513 described below. The image-data-of-affected-part field 910 stores image data of the affected part obtained by the image capturing for recording or address information regarding an address where the image data of the affected part is stored. The evaluation information field 911 stores information indicating a result of evaluation of the affected region. The inclination information field 912 stores inclination information regarding the imaging apparatus 200 in the image capturing for recording.

If the object information 900 corresponding to the target patient ID is not stored in step S537, the CPU 310 adds information to the posture icon field 904, the image-data-of-posture field 905, and the first inclination information field 906 in the posture information 903 of the generated object information 900, and stores the resulting information in the storage device 312. Specifically, to add information to the posture icon field 904, first, based on the image data of the posture received by the auxiliary calculation device 317 in step S531, the CPU 310 determines to which of the posture icons 921 to 924 illustrated in FIG. 9B the posture of the object corresponds. Next, the CPU 310 stores the posture icon or the identification information regarding the posture icon in the posture icon field 904. The CPU 310 also stores the image data of the posture received in step S531 in the image-data-of-posture field 905. Further, the CPU 310 stores the inclination information regarding the imaging apparatus 200 when the posture is captured that is received in step S531 in the first inclination information field 906.

On the other hand, if the object information 900 corresponding to the target patient ID is stored in step S537, this means that an image of the affected part has been captured in the past and pieces of information are already stored in the posture information 903 and the affected part information 908 of the object information 900. Thus, the processing proceeds to step S538.

In step S538, the CPU 310 of the information processing apparatus 300 transmits the information indicating the result of extraction of the affected region and the information regarding the size of the affected region to the imaging apparatus 200 via the communication device 313. In the present exemplary embodiment, the CPU 310 transmits the image data obtained by superimposing the information indicating the result of extraction of the affected region and the information regarding the size of the affected region on the image data of the affected part that is generated in step S534 to the imaging apparatus 200.

To notify the user of the posture of the object taken when the affected part has been captured in the past, the CPU 310 transmits the posture information 903 of the object information 900 to the imaging apparatus 200 via the communication device 313. Specifically, the CPU 310 transmits the posture icon, the image data of the posture, the inclination information regarding the imaging apparatus 200 when the posture is captured, and the inclination information regarding the imaging apparatus 200 in the image capturing for recording. In a case where the CPU 310 transmits, multiple times during the live view, the image data obtained by superimposing the information indicating the result of extraction of the affected region and the information regarding the size of the affected region on the image data of the affected part, the CPU 310 transmits the posture information 903 only the first time. Alternatively, the CPU 310 may transmit the inclination information regarding the imaging apparatus 200 in the live view that is received in step S531. If the object information 900 corresponding to the target patient ID is not stored in step S537 because the image capturing for recording has not been performed in the past, information is not stored in the second inclination information field 907. Thus, the inclination information regarding the imaging apparatus 200 in the image capturing for recording is not transmitted.

Then, the processing performed by the imaging apparatus 200 will be described.

In step S509, the system control circuit 220 of the imaging apparatus 200 receives, via the communication device 219, the image data obtained by superimposing the information indicating the result of extraction of the affected region and the information regarding the size of the affected region on the image data of the affected part that is transmitted from the information processing apparatus 300. The system control circuit 220 also receives, via the communication device 219, the posture icon, the image data of the posture, the inclination information regarding the imaging apparatus 200 when the posture is captured, and the inclination information regarding the imaging apparatus 200 in the image capturing for recording that are transmitted from the information processing apparatus 300.

In step S510, the system control circuit 220 displays, on the display device 223, the image data obtained by superimposing the information indicating the result of extraction of the affected region and the information regarding the size of the affected region on the image data of the affected part. The information indicating the result of extraction of the affected part is thus displayed in a superimposed manner on the image data in the live view, whereby the user can confirm whether the estimated area and the area of the affected region are appropriate, and then proceed to the image capturing for recording.

The system control circuit 220 also displays, on the display device 223, at least any posture information among the posture icon, the image data of the posture, and the inclination information regarding the imaging apparatus 200 when the posture is captured that are received. The user is thus notified of the posture information regarding the object obtained when the image of the affected part has been captured in the past. The system control circuit 220 may display the inclination information regarding the imaging apparatus 200 in the image capturing for recording and the inclination information regarding the imaging apparatus 200 in the live view.

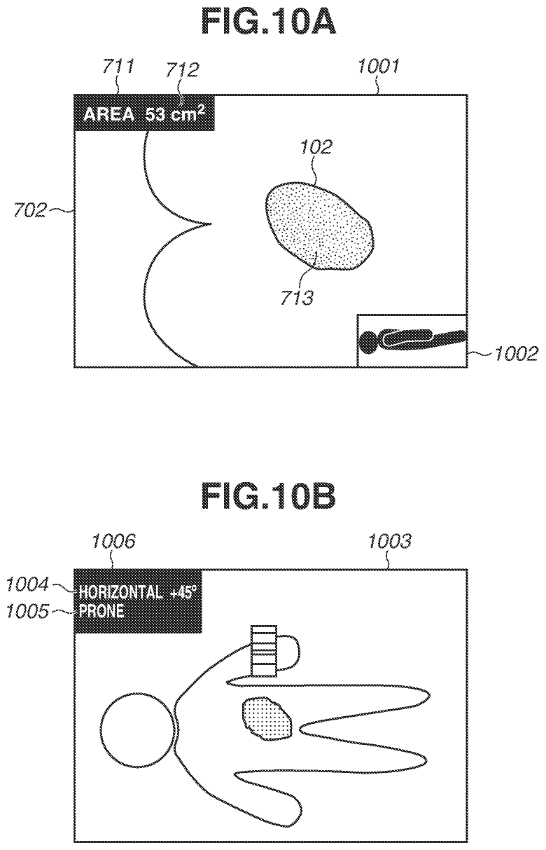

FIGS. 10A and 10B are diagrams illustrating examples of image data including the posture information. Portions similar to those in FIGS. 7A and 7B are indicated by the same reference numerals, and the description of the similar portions is appropriately omitted.

An image 1001 illustrated in FIG. 10A is an example of display of image data obtained by superimposing a posture icon 1002 on the image 702 illustrated in FIG. 7B.

The system control circuit 220 displays, on the display device 223, the image 1001 obtained by superimposing the posture icon 1002, which is based on the posture icon or the identification information regarding the posture icon that is received in step S509, on the image 702 illustrated in FIG. 7B.

In FIG. 7B, the posture icon 1002 functions as a button on which the user can perform a touch operation through the touch panel that also serves as the display device 223. In response to the touch operation by the user on the posture icon 1002, the system control circuit 220 transitions the screen and displays an image 1003 illustrated in FIG. 10B.

The image 1003 illustrated in FIG. 10B is an example of display of the image data of the posture. At the upper left corner of the image 1003, a label 1006 including inclination information 1004 and a character string 1005 is displayed in white characters on a black background.

The system control circuit 220 displays, on the display device 223, the image 1003 obtained by superimposing the label 1006 on the image data of the posture received in step S509. Based on the inclination information regarding the imaging apparatus 200 when the posture is captured that is received in step S509, the system control circuit 220 displays the inclination information 1004. In a case where the posture information received in step S509 includes the character information representing the posture, the system control circuit 220 displays the character string 1005 of the label 1006 based on the character information regarding the posture.

As described above, before the image of the affected part is captured for recording, the user is notified of the posture information regarding the object obtained when the image of the affected part of the same object has been captured in the past, whereby the user can grasp the posture taken when the image of the affected part of the object has been captured in the past. Thus, the user can ask the object to take the same posture as the posture taken when the image of the affected part has been captured in the past, and thereby can appropriately capture the image of the affected part of the object.

Specifically, the posture icon 1002 schematically illustrating the posture of the object is displayed, whereby the user can immediately grasp the posture of the object taken when the image of the affected part of the object has been captured in the past. The image 1003 obtained by capturing the posture of the object is also displayed, whereby the user can accurately grasp the posture of the object taken when the image of the affected part of the object has been captured in the past. Further, the inclination information 1004 regarding the imaging apparatus 200 is displayed, whereby the user can grasp the inclination of the imaging apparatus 200 when the posture is captured. However, an image in which the posture information is to be displayed is not limited to the images illustrated in FIGS. 10A and 10B, and may be any image as long as the user can grasp the posture of the object.

The system control circuit 220 may display the inclination information regarding the imaging apparatus 200 in the image capturing for recording that is received in step S509. The user references the displayed inclination information and thereby can capture the image of the affected part at an inclination similar to that when the image of the affected part has been captured in the past. Thus, the user can cause the imaging apparatus 200 to face the surface of the affected part.

At this time, the system control circuit 220 may display the inclination information regarding the imaging apparatus 200 in the live view that is generated in step S507 or received in step S509. In this case, the user can reference the inclination of the imaging apparatus 200 at the current moment and thus can match the current inclination to the inclination when the image of the affected part has been captured in the past. The system control circuit 220 may display information regarding a difference between the inclination information regarding the imaging apparatus 200 in the image capturing for recording and the inclination information regarding the imaging apparatus 200 in the live view. The information regarding the difference may be generated by the system control circuit 220 of the imaging apparatus 200, or may be generated by the information processing apparatus 300 and received by the imaging apparatus 200.

In step S511, the system control circuit 220 determines whether an image capturing instruction issued by the user pressing a shutter release button included in the operation unit 224 is received.

If the image capturing instruction is received (YES in step S511), in step S512 and the subsequent steps, the processing proceeds to the process of capturing the image of the affected part for recording. On the other hand, if the image capturing instruction is not received (NO in step S511), the processing returns to step S503, and the processes of step S503 and the subsequent steps are performed. Thus, the processes of steps S503 to S511 are repeated until the image capturing instruction is received, whereby the imaging apparatus 200 continuously transmits the image data in the live view to the information processing apparatus 300. Every time the imaging apparatus 200 transmits the image data, the imaging apparatus 200 receives, from the information processing apparatus 300, the image data obtained by superimposing the information indicating the result of extraction of the affected region and the information regarding the size of the affected region on the image data of the affected part.

In step S512, the AF control circuit 218 performs an AF process for controlling the driving of the lens group 212 so that the object comes into focus. This process is similar to the process of step S503.

In step S513, in response to an image capturing instruction from the user, the image capturing unit 211 captures an image of the object. Specifically, the image capturing unit 211 captures a still image of the affected part for recording.

If it is determined in step S537 that the object information 900 corresponding to the target patient ID is not stored, the system control circuit 220 may prompt the user to first capture an image of the affected part for recording and then capture an image of the posture of the object. Specifically, the system control circuit 220 adjusts the magnification of the image capturing unit 211 so that, after the affected part is captured, the entire body of the object is captured. Then, the user performs image capturing. In a case where the posture of the object is thus automatically captured, the process of capturing the posture of the object in step S502 can be omitted. Information indicating that the object information 900 corresponding to the target patient ID is not stored can be received from the information processing apparatus 300 in step S509.

In step S514, the image processing circuit 217 acquires data of the captured image and performs a development process and a compression process on the image data, thereby generating image data based on the JPEG standard, for example. This process is similar to the process of step S505. However, to give priority to accuracy of the measurement of the affected region, it is desirable that the image processing circuit 217 perform a resizing process for resizing the image data to a larger size than or the same size as the size of the image data in step S505. For example, the size of the image data subjected to the resizing process is approximately 4.45 megabytes in the case of 1440 pixels×1080 pixels in 4-bit RGB colors. However, the size of the image data to be subjected to the resizing process is not limited to the above.

In step S515, the system control circuit 220 generates distance information regarding the distance to the object. This process is similar to the process of step S506.

In step S516, based on the inclination information output from the inclination detection device 225, the system control circuit 220 generates inclination information regarding the imaging apparatus 200 in the image capturing for recording. This process is similar to the process of step S507.

In step S517, the system control circuit 220 transmits the image data of the affected part subjected to the resizing process in step S514, the distance information regarding the distance to the object that is generated in step S515, and the inclination information regarding the imaging apparatus 200 in the image capturing for recording that is generated in step S516, to the information processing apparatus 300 via the communication device 219.

Then, the processing performed by the information processing apparatus 300 will be described.

In step S541, the CPU 310 of the information processing apparatus 300 receives, via the communication device 313, the image data of the affected part, the distance information regarding the distance to the object, and the inclination information regarding the imaging apparatus 200 in the image capturing for recording that are transmitted from the imaging apparatus 200.

In step S542, using the auxiliary calculation device 317, the CPU 310 extracts an affected region from the received image data of the affected part (segments the affected region and another region). This process is similar to the process of step S532.

In step S543, the calculation device 311 of the CPU 310 calculates the area of the affected region as information regarding the size of the extracted affected region. This process is similar to the process of step S533.

In step S544, the calculation device 311 calculates evaluation information regarding the affected region. Specifically, based on the length on the focal plane corresponding to one pixel on the image that is obtained in step S543, the calculation device 311 calculates the lengths of the major axis and the minor axis of the extracted affected region and the area of a rectangle circumscribing the affected region. DESIGN-R as an evaluation indicator for a pressure ulcer defines that the size of a pressure ulcer is to be obtained by measuring the value of the product of the major axis and the minor axis. The image processing system 1 according to the present exemplary embodiment analyzes the major axis and the minor axis and thereby can secure compatibility with data measured by DESIGN-R in the past. Since DESIGN-R does not provide a strict definition, a plurality of calculation methods is mathematically possible as the calculation method for calculating the major axis and the minor axis.

As a first example of the calculation method for calculating the major axis and the minor axis, the calculation device 311 calculates a rectangle having the smallest area (a minimum bounding rectangle) among rectangles circumscribing the affected region. Next, the calculation device 311 calculates the lengths of the long side and the short side of the rectangle, and uses the length of the long side as the major axis and the length of the short side as the minor axis in calculation. Then, based on the length on the focal plane corresponding to one pixel on the image that is obtained in step S543, the calculation device 311 calculates the area of the rectangle.

As a second example of the calculation method for calculating the major axis and the minor axis, the calculation device 311 selects the maximum Feret's diameter that is the maximum caliper length as the major axis, and selects the minimum Feret's diameter as the minor axis. Alternatively, the calculation device 311 may select the maximum Feret's diameter that is the maximum caliper length as the major axis, and select a length measured in a direction orthogonal to the axis of the maximum Feret's diameter as the minor axis.

As the calculation method for calculating the major axis and the minor axis, any method can be selected based on compatibility with conventional measurement results.

The process of calculating the lengths of the major axis and the minor axis of the affected region and the area of the rectangle is not executed on the image data received in step S531. The live view is intended to enable the user to confirm the result of extraction of the affected region. Thus, during the live view, an image analysis process corresponding to step S544 on the image data received in step S531 is omitted, thereby reducing the processing time.

In step S545, the image processing circuit 217 generates image data obtained by superimposing information indicating a result of extraction of the affected region and information regarding the size of the affected region on the image data from which the affected region is to be extracted. The information regarding the size of the affected region in this step includes the evaluation information regarding the affected region, such as the major axis and the minor axis of the affected region.

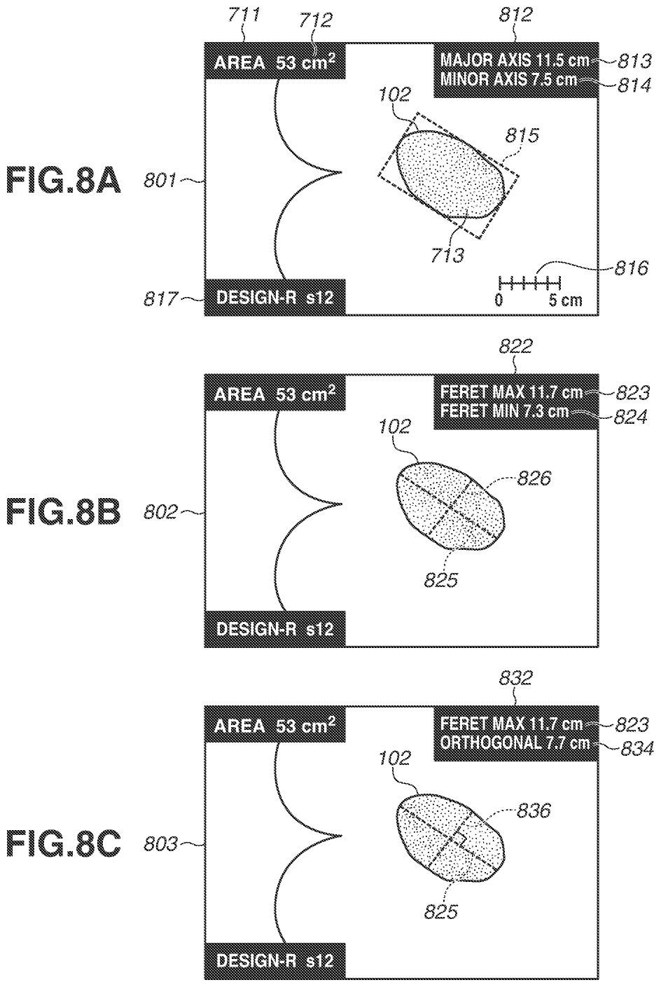

FIGS. 8A, 8B, and 8C are diagrams illustrating the method for superimposing the information indicating the result of extraction of the affected region and the information regarding the size of the affected region including the major axis and the minor axis of the affected region on the image data. Since a plurality of pieces of information regarding the size of the affected region is expected, a description is given with reference to FIGS. 8A to 8C.

An image 801 illustrated in FIG. 8A is obtained using the minimum bounding rectangle as the calculation method for calculating the major axis and the minor axis. As with FIG. 7B, at the upper left corner of the image 801, as the information regarding the size of the affected region, the label 711 is superimposed in which the character string 712 indicating the area of the affected region is displayed in white characters on a black background.

At the upper right corner of the image 801, as the information regarding the size of the affected region, a label 812 is superimposed in which the major axis and the minor axis calculated based on the minimum bounding rectangle are displayed.

The label 812 includes character strings 813 and 814. The character string 813 indicates the length of the major axis (in centimeters (cm)). The character string 814 indicates the length of the minor axis (in centimeters). In the image 801, a rectangular frame 815 representing the minimum bounding rectangle is superimposed on the affected region. The rectangular frame 815 is superimposed together with the lengths of the major axis and the minor axis, whereby the user can confirm in which portion in the image the lengths are measured.

At the lower right corner of the image 801, a scale bar 816 is superimposed. The scale bar 816 is used to measure the size of the affected part 102, and the size of the scale bar 816 relative to the image data is changed based on the distance information. Specifically, the scale bar 816 is a bar graduated up to 5 cm at 1-cm intervals based on the length on the focal plane corresponding to one pixel on the image that is obtained in step S543, and corresponds to the size on the focal plane of the imaging apparatus 200, i.e., on the object 101. The user references the scale bar 816 and thereby can grasp the size of the object 101 or the affected part 102.

At the lower left corner of the image 801, an indicator 817 for Size evaluation of DESIGN-R is superimposed. In the indicator 817 for Size evaluation of DESIGN-R, based on a numerical value obtained by measuring, in centimeters, the major axis and the minor axis (the maximum diameter orthogonal to the major axis) of the extent of skin injury and by multiplying the major axis and the minor axis, Size is classified into the above-described seven levels. In the present exemplary embodiment, the indicator 817 obtained by replacing the major axis and the minor axis with values output using respective calculation methods for calculating the major axis and the minor axis is superimposed.

An image 802 illustrated in FIG. 8B is obtained using the maximum Feret's diameter as the major axis and the minimum Feret's diameter as the minor axis. At the upper right corner of the image 802, a label 822 is superimposed in which a character string 823 indicating the length of the major axis and a character string 824 indicating the length of the minor axis are displayed. In the affected region of the image 802, an additional line 825 corresponding to the measurement position of the maximum Feret's diameter and an additional line 826 corresponding to the minimum Feret's diameter are displayed. The additional lines 825 and 826 as well as the character strings 823 and 824 indicating the lengths of the major axis and the minor axis are superimposed, whereby the user can confirm in which portion in the image the lengths are measured.

In an image 803 illustrated in FIG. 8C, the major axis is the same as that in the image 802, but the minor axis is not the minimum Feret's diameter and is a length measured in a direction orthogonal to the axis of the maximum Feret's diameter. At the upper right corner of the image 803, a label 832 is superimposed in which the character string 823 indicating the length of the major axis and a character string 834 indicating the length of the minor axis are displayed. In the affected region of the image 803, the additional line 825 corresponding to the measurement position of the maximum Feret's diameter and an additional line 836 corresponding to the length measured in the direction orthogonal to the axis of the maximum Feret's diameter are displayed.

The various pieces of information to be superimposed on the image data illustrated in FIGS. 8A to 8C may be any one of the pieces of information or a combination of a plurality of the pieces of information. The user may be allowed to select information to be displayed. The images illustrated in FIGS. 7A, 7B, 8A, 8B, and 8C are merely examples, and the display forms, the display positions, the sizes, the fonts, the font sizes, or the font colors of the pieces of information regarding the sizes of the affected part 102 and the affected region, or the positional relationships between the pieces of information can be changed to meet various conditions.

In step S546, the CPU 310 of the information processing apparatus 300 transmits the information indicating the result of extraction of the affected region and the information regarding the size of the affected region to the imaging apparatus 200 via the communication device 313. In the present exemplary embodiment, the CPU 310 transmits the image data obtained by superimposing the information indicating the result of extraction of the affected region and the information regarding the size of the affected region on the image data of the affected part that is generated in step S545 to the imaging apparatus 200.

In step S547, the CPU 310 reads the patient ID from the image data of the barcode tag. If the patient ID is already read in step S535, the process of step S547 can be omitted.

In step S548, the CPU 310 checks the read patient ID against the patient ID of the object registered in advance, thereby acquiring information regarding the name of the object. If the information regarding the name of the object is already acquired in step S536, the process of step S548 can be omitted.

In step S549, the CPU 310 adds information to the image-capturing-date-and-time field 909, the image-data-of-affected-part field 910, the evaluation information field 911, and the inclination information field 912 in the affected part information 908 of the object information 900 corresponding to the target patient ID. In step S550, the CPU 310 stores the resulting information in the storage device 312.

Specifically, the CPU 310 stores information regarding the date and time of the image capturing performed in step S513 in the image-capturing-date-and-time field 909. The CPU 310 also stores the image data of the affected part received in step S541 in the image-data-of-affected-part field 910. The CPU 310 also stores the evaluation information calculated in step S544 in the evaluation information field 911. The CPU 310 also stores the inclination information regarding the imaging apparatus 200 in the image capturing for recording that is received in step S541 in the inclination information field 912. As described with respect to the object information 900 in FIG. 9A, based on the inclination information stored in the inclination information field 912, the CPU 310 can store or update the inclination information in the second inclination information field 907 in the posture information 903.

If the object information corresponding to the target patient ID is not stored in the storage device 312, the CPU 310 generates object information corresponding to the patient ID and the information regarding the name of the object, and stores information in the posture information 903 and the affected part information 908 of the object information 900.

If the object information corresponding to the target patient ID is stored in the storage device 312, the CPU 310 may determine whether the image data already stored in the image-data-of-posture field 905 and the image data of the posture obtained in step S502 in the current image capturing match each other. These pieces of image data matching each other means that the postures of the object included in both pieces of image data are the same. Thus, for example, if the object included in one of the pieces of image data takes a prone posture and the object included in the other image data takes a recumbent posture, the CPU 310 determines that the pieces of image data do not match. If the pieces of image data do not match, the CPU 310 updates the image data already stored in the image-data-of-posture field 905 with the image data of the posture obtained in step S502 in the current image capturing, and stores the updated image data. The CPU 310 may update and store not only the image data of the posture, but also at least either of the posture icon field 904 and the first inclination information field 906 in the posture information 903.

Then, the processing performed by the imaging apparatus 200 will be described.

In step S518, the system control circuit 220 of the imaging apparatus 200 receives, via the communication device 219, the image data obtained by superimposing the information indicating the result of extraction of the affected region and the information regarding the size of the affected region on the image data of the affected part that is transmitted from the information processing apparatus 300.

In step S519, the system control circuit 220 displays, on the display device 223, the image data obtained by superimposing the information indicating the result of extraction of the affected region and the information regarding the size of the affected region on the received image data of the affected part, for a predetermined time. In this step, the system control circuit 220 displays any of the images 801 to 803 illustrated in FIGS. 8A to 8C, and if the predetermined time elapses, the processing returns to step S503.

As described above, in the present exemplary embodiment, in a case where the user captures an image of an affected part using the imaging apparatus 200, the user is notified of posture information regarding an object obtained when an image of the affected part of the same object has been captured in the past, whereby the user can capture the image of the image of the affected part by setting the posture of the object to the same posture as that when the object has been captured in the past. Thus, the user can capture an image with which the user can compare progress more accurately.

In the present exemplary embodiment, DESIGN-R (registered trademark) is used as an evaluation indicator of a pressure ulcer. However, the present invention is not limited to this. Alternatively, another evaluation indicator such as the Bates-Jensen Wound Assessment Tool (BWAT), the Pressure Ulcer Scale for Healing (PUSH), or the Pressure Sore Status Tool (PSST) may also be used.

### (First Modification)

In step S502 in the flowchart in FIG. 5A, a case has been described where the image of the posture of the object is captured. However, the present invention is not limited to this case. For example, a configuration may be employed in which, in step S502, the imaging apparatus 200 allows the user to select the posture of the object. Specifically, in step S502, the system control circuit 220 displays the posture icons 921 to 924 illustrated in FIG. 9B or character information indicating postures in a selectable manner on the display device 223. Thus, the user can select a posture icon or character information corresponding to the posture of the object. In step S508, the system control circuit 220 transmits the posture icon (including identification information regarding the posture icon) or the character information selected by the user to the information processing apparatus 300.

The user is thus allowed to select the posture of the object, whereby it is possible to easily identify the posture of the object. Further, the process of transmitting and receiving the image data of the posture can be omitted. Thus, it is possible to reduce a processing load on the image processing system 1.

### (Second Modification)

In step S538 in the flowchart in FIG. 5A, a case has been described where the posture information 903 of the object information 900 is transmitted to the imaging apparatus 200 to notify the user of the posture of the object taken when the image of the affected part has been captured in the past. However, the present invention is not limited to this case. For example, if it is determined in step S537 that the object information 900 corresponding to the target patient ID is not stored in the storage device 312, the CPU 310 need not transmit the posture information 903 to the imaging apparatus 200. This is because, in the case where the object information 900 corresponding to the target patient ID is not stored in the storage device 312 in step S537, the object is captured for the first time this time, and therefore, it is less necessary to notify the user of the posture of the object taken when the image of the affected part has been captured in the past.

### (Third Modification)

In step S510 in the flowchart in FIG. 5, a case has been described where the system control circuit 220 displays, on the display device 223, the posture of the object taken when the image of the affected part has been captured in the past. However, the present invention is not limited to this case. For example, the system control circuit 220 may notify the user of the posture of the object taken when the image of the affected part has been captured in the past by sound using a sound device (not illustrated).

While the present invention has been described above together with various exemplary embodiments and modifications, the present invention is not limited to the above exemplary embodiments and modifications and can be changed within the scope of the present invention. The above exemplary embodiments and modifications may be combined together when appropriate. For example, a target analyzed by the information processing apparatus 300 is not limited to an affected part, and may be an object included in image data.

The present invention is not limited to the above exemplary embodiments, and can be changed and modified in various manners without departing from the spirit and the scope of the present invention. Thus, the following claims are appended to publicize the scope of the present invention.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An imaging apparatus comprising:
an image capturing unit; and
a control unit configured to perform control to, in a case where posture information regarding an object obtained when an image of an affected part of the object has been captured in the past is acquired and the image capturing unit captures an image of the affected part of the object, notify a user of the posture information regarding the object and inclination information regarding the imaging apparatus.

2. The imaging apparatus according to claim 1, wherein the control unit displays the posture information regarding the object and the inclination information regarding the imaging apparatus on a display device.

3. The imaging apparatus according to claim 2, wherein the control unit displays at least any one of a display item schematically illustrating a posture of the object, image data obtained by capturing an image of the posture of the object, inclination information regarding the imaging apparatus obtained when the image of the posture of the object is captured, and character information representing the posture of the object in a character, as the posture information on the display device.

4. The imaging apparatus according to claim 2,
wherein the affected part is a pressure ulcer, and
wherein the posture information includes information that allows identification of at least any one of a prone posture, a recumbent posture, and a sitting posture of the object.

5. The imaging apparatus according to claim 2, wherein the control unit displays, on the display device, the posture information regarding the object and the inclination information regarding the imaging apparatus in a superimposed manner on image data in live view captured by the image capturing unit.

6. The imaging apparatus according to claim 2, wherein the control unit transitions a screen on which image data in live view captured by the image capturing unit is displayed to a different screen in response to a user operation, thereby displaying the posture information regarding the object and the inclination information regarding the imaging apparatus on the display device.

7. The imaging apparatus according to claim 5, wherein the image data in the live view captured by the image capturing unit is image data transmitted from the imaging apparatus to an external apparatus, subjected to image processing by the external apparatus, and then received from the external apparatus.

8. The imaging apparatus according to claim 1, further comprising a communication unit configured to transmit identification information regarding the object to an external apparatus,
wherein the control unit controls the communication unit to transmit the identification information to the external apparatus and receive the posture information and the inclination information associated with the identification information from the external apparatus.

9. The imaging apparatus according to claim 1, further comprising a communication unit configured to, in a case where the image capturing unit captures the image of the affected part of the object, transmit the posture information regarding the object and the inclination information regarding the imaging apparatus to an external apparatus,
wherein the control unit receives, from the external apparatus via the communication unit, the posture information and the inclination information transmitted from the communication unit and stored in the external apparatus.

10. The imaging apparatus according to claim 9, wherein the communication unit transmits image data obtained by the image capturing unit capturing an image of a posture of the object to the external apparatus.

11. The imaging apparatus according to claim 9, wherein the communication unit transmits posture information selected from a plurality of pieces of posture information by the user to the external apparatus.

12. An information processing apparatus comprising:
a communication unit configured to receive identification information regarding an object from an imaging apparatus and transmit, to the imaging apparatus, posture information regarding the object obtained when an image of an affected part of the object has been captured in the past and inclination information regarding the imaging apparatus that are associated with the identification information.

13. The information processing apparatus according to claim 12,
wherein the identification information regarding the object and the posture information regarding the object are stored in association with each other in a storage device,
wherein the communication unit receives the identification information regarding the object and the posture information regarding the object from the imaging apparatus, and
wherein, in a case where the posture information stored in the storage device in association with the same identification information as the identification information received by the communication unit does not match the posture information received by the communication unit, the control unit updates the posture information stored in the storage device with the posture information received by the communication unit.

14. The information processing apparatus according to claim 12,
wherein the communication unit receives the identification information regarding the object and posture information regarding the object from the imaging apparatus, and
wherein in a case where object information corresponding to the identification information received by the communication unit is not stored in a storage device, the control unit stores the identification information and the posture information received by the communication unit in association with each other in the storage device.

15. An image processing system comprising:
an information processing apparatus including:
a communication unit configured to receive identification information regarding an object from an imaging apparatus and transmit, to the imaging apparatus, posture information regarding the object obtained when an image of an affected part of the object has been captured in the past and inclination information regarding the imaging apparatus that are associated with the identification information; and
the imaging apparatus including:
an image capturing unit; and
a control unit configured to, in a case where the image capturing unit captures an image of the affected part of the object, notify a user of the posture information regarding the object and the inclination information regarding the imaging apparatus that are transmitted from the communication unit.

16. A control method for controlling an imaging apparatus, the control method comprising:
acquiring posture information regarding an object obtained when an image of an affected part of the object has been captured in the past, and inclination information regarding the imaging apparatus;
performing control to notify a user of the posture information regarding the object and the inclination information regarding the imaging apparatus; and
capturing an image of the affected part of the object.

17. A control method for controlling an information processing apparatus, the control method comprising:
receiving identification information regarding an object from an imaging apparatus; and
transmitting, to the imaging apparatus, posture information regarding the object obtained when an image of an affected part of the object has been captured in the past and inclination information regarding the imaging apparatus that are associated with the identification information.

18. A control method for controlling an image processing system including an imaging apparatus and an information processing apparatus, the control method comprising:
transmitting identification information regarding an object from the imaging apparatus to the information processing apparatus;
transmitting, from the information processing apparatus to the imaging apparatus, posture information regarding the object obtained when an image of an affected part of the object has been captured in the past and inclination information regarding the imaging apparatus that are associated with the identification information;
performing control to notify a user of the posture information regarding the object and the inclination information regarding the imaging apparatus that are acquired by the imaging apparatus; and
capturing an image of the affected part of the object.
